(19) United States
(12) Patent Application Publication
(10) Pub. No.: US 2025/0122265 A1
(43) Pub. Date: Apr. 17, 2025

(54) RECOMBINANT HUMAN CD5L PROTEIN, ACTIVE FRAGMENTS OR PEPTIDES DERIVED THEREOF AND PHARMACEUTICAL COMPOSITION COMPRISING THE RECOMBINANT HUMAN CD5L PROTEIN, ACTIVE FRAGMENTS OR PEPTIDES DERIVED THEREOF FOR THE TREATMENT OF ACUTE INFECTIOUS DISEASES,INFLAMMATORY DISEASES AND SEPSIS

(71) Applicants: IBMC (INSTITUTO DE BIOLOGIA MOLECULAR E CELULAR), PORTO (PT); INVIGATE GMBH, JENA (DE)

(72) Inventors: Alexandre Valentim XAVIER MOURÃO DO CARMO, PORTO (PT); Liliana Sofia DA SILVA MARTINS DE OLIVEIRA, PORTO (PT); Sebastian KRAUSE, JENA (DE)

(21) Appl. No.: 18/728,718

(22) PCT Filed: Jan. 25, 2022
(86) PCT No.: PCT/PT2022/050004

§ 371 Date: Jul. 12, 2024

(51) Int. Cl. C07K 14/705; A61K 38/00

(52) U.S. Cl. CPC C07K 14/70596; A61K 38/00; C07K 2319/21

(57) ABSTRACT

The present invention describes a recombinant form of the human circulating scavenger protein CD5L, as well as active fragments or peptides derived thereof.

The invention further describes a pharmaceutical composition comprising the referred recombinant human CD5L protein, or one or more active fragments or peptides derived thereof, or one or more nucleic acids encoding full-length human CD5L or active fragments thereof.

It is also an object of this invention the use of the pharmaceutical composition comprising the recombinant human CD5L protein, or one or more active fragments or peptides derived thereof, in the treatment of acute infectious diseases, inflammatory diseases and sepsis, in the absence of association with complementary drugs.

The invention solves state-of-the-art problems regarding the need to identify new therapeutic alternatives for the treatment of inflammation and sepsis, in addition to providing an advantage in combating the emergence of antibiotic resistance.

### TECHNICAL FIELD

The present invention describes a recombinant human CD5L protein, as well as active fragments or peptides derived thereof.

It is also an object of the present invention a pharmaceutical composition comprising the referred recombinant human CD5L protein, or one or more active fragments or peptides derived thereof, or one or more nucleic acids encoding full-length human CD5L or active fragments thereof for the treatment of acute infectious diseases, inflammatory diseases and sepsis.

### BACKGROUND ART

Sepsis can be defined as a systemic inflammatory syndrome that is characterized by patients having very high heart and respiratory rates, extremely high or low body temperatures and high leukocyte counts. This condition results from the disproportionate production of pro-inflammatory mediators triggered by the microbial foci that cause the initial infection. Uncontrolled inflammation can lead to septic shock, which can be more threatening than the infection itself.

Sepsis affects millions of patients worldwide, and data from 2017 estimated the occurrence of 49 million cases of sepsis in one year, with a death toll of 11 million, having thus a mortality rate of 22.5%, and representing nearly 20% of all deaths in the world (Rudd et al., 2020). Sepsis is also one of the leading causes of hospital deaths, and whereas in less severe cases mortality is around 30%, in cases of septic shock it can reach 70%.

The superfamily of proteins with extracellular scavenger receptor cysteine-rich (SRCR) domains includes a diverse group with more than 20 membrane-bound or secreted proteins present in a wide range of multicellular species. Different SRCR proteins play varied roles in cell differentiation, homeostasis or apoptosis; however, only now some key characteristics are emerging as distinctive of this family, such as their anti-microbial and anti-inflammatory properties.

CD6 and CD163 (expressed on T cells and macrophages, respectively), DMBT1 (widely expressed) and CD5L and SSC5D (circulating blood proteins) are all molecular sensors of Gram-positive and Gram-negative bacteria (Bessa Pereira et al., 2016; Fabriek et al., 2009; Holmskov et al., 1999; Sarrias et al., 2007; Sarrias et al., 2005).

Since it is already known that phylogenetically older SRCR proteins, such as MARCO, SR-AI and SCARA-5, contribute to the recognition and elimination of bacteria (Elomaa et al., 1995; Hampton et al., 1991), the concept arises that SRCRs constitute an autonomous family of pattern recognition receptors (PRR). However, unlike other groups of PRRs, such as Toll-like receptors and NOD-type receptors, SRCR proteins can actively contribute to the resolution of inflammation.

In this sense, given the unique combined anti-inflammatory and antimicrobial roles of SRCR proteins, the CD5L protein (CD5 antigen-like protein, also known as AIM—apoptosis inhibitor expressed by macrophages, Spα—soluble protein alpha, or Api6—apoptosis inhibitor 6), emerges as a therapeutic alternative for the treatment of infectious and inflammatory diseases, causing minimal toxic effects while promoting its curative role.

Because it is considered an emerging component in the immunological effector scenario, CD5L has been the object of numerous studies that explore its role in immune and inflammatory responses.

The article “The macrophage soluble receptor AIM/Api6/CD5L displays a broad pathogen recognition spectrum and is involved in early response to microbial aggression”, by Martinez and colleagues (Martínez et al., 2014), points out that CD5L is capable of binding and aggregating a wide spectrum of microbial agents (bacteria and fungi), thus potentially playing a role in the initial immune response to microbial agents. Furthermore, in in vitro cultures using ex-vivo spleen macrophages the protein also showed immunomodulatory properties that were interpreted as being potentially useful in protecting tissue damage from local inflammation as well as generalized damage, that could be sepsis and septic shock syndrome.

In the study by Sanjurjo and colleagues, entitled “AIM/CD5L: a key protein in the control of immune homeostasis and inflammatory disease” (Sanjurjo et al., 2015b), it is proposed to use the CD5L protein as a diagnostic and prognostic biomarker of various pathological aspects, due to its presence in human fluids and its versatility (with multiple activities in leukocytes, adipocytes and epithelial cells). Plasma CD5L levels are altered in several inflammatory conditions, such as sepsis, indicating that the said protein can be used as a biomarker of diseases. However, no continuation of this study was carried out regarding its anti-inflammatory potential.

The same authors also published the article “The human CD5L/AIM-CD36 axis: A novel autophagy inducer in macrophages that modulates inflammatory responses” (Sanjurjo et al., 2015a), which highlights the role of CD5L in the regulation of homeostasis, showing that this protein activates autophagy of macrophages through the surface receptor CD36. Although it points to the immunomodulatory capacity of the CD5L protein, this study does not suggest the use of the protein as a therapeutic agent.

In the study by Gao and colleagues published in the article “Therapeutic targeting of apoptosis inhibitor of macrophage/CD5L in sepsis” (Gao et al., 2019), the role of CD5L in modulating the immune and the inflammatory responses in cases of sepsis is assessed. Contrary to the invention to be protected, the authors of this publication show that supplementation of recombinant CD5L at the same time of the induction of sepsis by cecal ligation and puncture (CLP) results in increased tissue damage, amplification of inflammation, increased bacteremia and worsening mortality, probably associated with the production of IL-10 (anti-inflammatory cytokine that inhibits the activity of cells, needed to eliminate the pathogen).

WO2018218231A1 describes an antagonist of the CD5L protein, such as an antibody or a fragment of antibody that neutralizes CD5L, for the treatment of cancer, immune deficiencies, or infections caused by pathogens; meanwhile, WO2017087708A1 describes methods for modulating or suppressing an immune response in individuals with chronic inflammatory diseases, or with autoimmune diseases, or inflammation-related cancers, by administering therapeutically effective amounts of different forms of CD5L. Both of these international patent documents describe situations opposite to those described in the present invention, or in different contexts, since CD5L antagonists (recombinant, homodimers or heterodimers) modulate or improve the immune response in an individual; and the CD5L protein itself (recombinant, homodimers or heterodimers) is shown to suppress chronic immune responses in an individual.

In contrast, according to the studies carried out on the present invention, the CD5L protein has been shown to be effective in treating acute inflammation and sepsis even in the absence of complementary drugs, which constitutes an additional advantage to combat the emergence of antibiotic resistance. Thus, the therapeutic use of the protein in its recombinant form, as well as one or more active fragments or peptides derived thereof, or one or more nucleic acids encoding full-length CD5L or active fragments thereof, is promising and can have a great impact on the clinical management of conditions that include intense inflammatory responses.

### SUMMARY OF INVENTION

The present invention describes, in a first aspect, a recombinant human CD5L protein, which is set forth in SEQ ID No. 3. The invention further discloses active fragments or peptides derived from the recombinant human CD5L protein, which are set forth in SEQ ID Nos. 4 to 6 and SEQ ID Nos. 7 to 9, respectively.

In a second aspect, the present invention relates to a pharmaceutical composition for the treatment of acute infectious diseases, inflammatory diseases and sepsis, comprising:

a therapeutically effective amount of an active principle selected from the group consisting of a recombinant human CD5L protein or one or more active fragments or one or more peptides derived thereof, or one or more nucleic acids encoding full-length human CD5L or active fragments thereof and a suitable excipient, diluent or carrier, wherein the suitable excipient, diluent or carrier is selected from the group consisting of a stabilizing agent or combinations thereof, a surfactant or combinations thereof, a buffering agent or combinations thereof, and an antioxidant or combinations thereof.

In a third aspect, this invention relates to the use of such pharmacological composition for treating acute infectious diseases, inflammatory diseases and sepsis, especially during an exacerbated life-threatening inflammatory syndrome, wherein the blood levels of C-reactive protein (CRP) in the patient reach a value of 50% above normal and the blood oxygen saturation levels drop below 80%.

### Technical Problem

Most studies and publications related to the CD5L protein point to its activity as a disease biomarker, since the plasma levels of this protein are altered in different inflammatory conditions. CD5L is indicated in these same studies, and correctly so, as an agent that suppresses an immune response.

In other words, this protein is an immunosuppressant that has been considered as a possible therapeutic tool for the treatment of chronic inflammation. It should not, according to the present invention, be considered as a tool to suppress the natural body's beneficial inflammatory response that occurs immediately after and in response to an infection.

Thus, in view of the need to identify new therapeutic alternatives which allow the treatment of the secondary pathological exacerbated inflammatory conditions that may follow the initial infection, such as a cytokine storm, sepsis or septic shock, a pharmaceutical composition comprising the recombinant human scavenger protein CD5L, as well as one or more active fragments or peptides derived thereof, or one or more nucleic acids encoding full-length CD5L or active fragments thereof, looks promising.

### Solution to Problem

The present invention solves the problems of the prior art by proposing a recombinant human CD5L protein, as well as active fragments or peptides derived thereof, and a pharmaceutical composition comprising the recombinant human CD5L protein, as well as one or more active fragments or peptides derived thereof, or one or more nucleic acids encoding full-length human CD5L or active fragments thereof, as an effective therapy to treat acute infectious diseases, inflammatory diseases and sepsis in the absence of complementary drugs.

Also, given that the CD5L protein is an endogenous human protein, the use of said CD5L protein in its recombinant form minimizes the possibility of toxic effects and contributes to the fight against the emergence of antibiotic resistance.

### Advantageous Effects of Invention

This invention describes a recombinant human CD5L protein, as well as active fragments or peptides derived thereof, and a pharmaceutical composition useful for the treatment of acute infectious diseases, inflammatory diseases and sepsis, which comprises the referred recombinant human CD5L protein, as well as one or more active fragments or peptides derived thereof, or one or more nucleic acids encoding full-length human CD5L or active fragments thereof. As it is a protein naturally expressed by macrophages, there is a low probability of adverse immune reactions directed against pharmaceutically administered CD5L.

Since nowadays antibiotics are the drugs of first choice in the treatment of infections caused by bacterial microorganisms that can originate sepsis, the pharmaceutical composition proposed in the present invention also contributes to the fight against microbial resistance, given that the CD5L protein proved to be effective even in the absence of other drugs.

### BRIEF DESCRIPTION OF DRAWINGS

In order to promote an understanding of the principles according to the modalities of the present invention, reference will be made to the modalities illustrated in the figures and the language used to describe them.

It should also be understood that there is no intention to limit the scope of the invention to the content of the figures and that modifications to the inventive features illustrated herein, as well as additional applications of the principles and embodiments illustrated, which would normally occur to a person skilled in the art having the possession of this description, are considered within the scope of the claimed invention.

FIG. 1A illustrates the strategy for the development of CD5L knockout (KO) mice using CRISPR/Cas9 technology.

FIG. 1B graphically illustrates the quantification of serum CD5L in wild-type (WT) and KO mice determined by ELISA assay.

FIG. 1C illustrates peritoneal cells from healthy WT or CD5L-KO mice stained for CD5L (scale bar, 10 μm).

FIG. 1D graphically illustrates the absolute cell number (panel a) and frequency (panel b) of leukocyte sub-populations in the spleen of healthy WT or CD5L-KO mice, measured by flow cytometry.

FIG. 1E graphically illustrates the absolute cell number (panel a) and frequency (panel b) of leukocyte sub-populations in the peritoneal cavity of healthy WT or CD5L-KO mice, measured by flow cytometry.

FIG. 1F graphically illustrates the survival percentage of WT and CD5L-KO mice injected intraperitoneally (IP) with a sub-lethal dose (5 mg/Kg of body mass) of lipopolysaccharide (LPS).

FIG. 1G graphically illustrates the survival percentage of WT and CD5L-KO mice submitted to cecal ligation and puncture (CLP) to induce medium-grade sepsis.

FIG. 2A graphically illustrates the counts of colony-forming units (CFU), grown under aerobic or anaerobic conditions, of samples obtained from the peritoneal cavity (panel a), blood (panel b), lung (panel c), liver (panel d) and kidney (panel e) of WT and CD5L-KO mice submitted to CLP to induce medium-grade sepsis.

FIG. 2B graphically illustrates the absolute cell number (panels a, b, d, f and h) and frequency (panels c, e, g and i) of leukocyte sub-populations (more specifically, neutrophils, total macrophages, Ly6C⁺ and CD206⁺ macrophages) in the peritoneal cavity of WT and CD5L-KO mice submitted to CLP to induce medium grade sepsis, measured by flow cytometry.

FIG. 2C graphically illustrates, using geometric mean statistics, the mean fluorescence intensity (MFI) of the Ly6G and CD11b markers expressed by neutrophils from the peritoneal cavity of WT and CD5L-KO mice submitted to medium-grade CLP.

FIG. 2D shows sections of renal, hepatic and pulmonary tissue from WT and CD5L-KO mice obtained 24 hours after medium-grade CLP and stained with hematoxylin and eosin (scale bar: 100 μm).

FIG. 2E graphically illustrates the pathology scores of kidney, liver and lung of WT and CD5L-KO mice 24 hours after medium-grade CLP, assessed by a double-blind analysis, in which 0—no sign of inflammation; 1—minimal inflammatory signs; 2—mild inflammation; 3—moderate to severe inflammation.

FIG. 2F graphically illustrates the quantification of cytokines and chemokines by ELISA assay in samples of the peritoneal cavity (panels a and b) and blood serum (panels c and d) collected at 6 and 24 hours after surgery from WT and CD5L-KO mice submitted to medium-grade CLP.

FIG. 3A graphically illustrates the quantification of CD5L in the peritoneal cavity (panel a) and in the blood serum (panel b) of C57BL/6 WT mice 6 and 24 hours after medium-grade sepsis (by CLP).

FIG. 3B graphically illustrates that recombinant mouse CD5L administered intravenously (IV) to CD5L-KO mice undergoing medium-grade CLP, at 2.5 mg/Kg of body mass, results in a physiological blood concentration of the protein (panel b), which is recruited to the site of infection in the peritoneum (panel a). It also shows that CD5L administered IV to WT mice upon medium-grade CLP results in an enhanced protective concentration of CD5L in the peritoneum (panel a) and blood (panel b).

FIG. 4A graphically illustrates the survival curve of C57BL/6 WT mice undergoing LPS-induced lethal sepsis that were treated with different dosages of recombinant mouse CD5L administered IP.

FIG. 4B graphically illustrates the survival curve of C57BL/6 WT mice undergoing CLP-induced lethal sepsis that were treated with different dosages of recombinant mouse or human CD5L administered IP.

FIG. 4C schematizes the protocol of CLP-induced sepsis and treatment with recombinant mouse CD5L administered IP in C57BL/6 WT mice, and the analysis of the immune response.

FIG. 4D graphically illustrates the CFU counts of bacteria, obtained at 6 and 24 hours after surgery, from the peritoneal cavity (panel a) and blood (panel b), grown in aerobic or anaerobic conditions, or from lung (panel c), liver (panel d) and kidney (panel e), grown in aerobiosis, of WT mice submitted to CLP to induce lethal-grade sepsis, and treated, or not-treated, with recombinant mouse CD5L administered IP.

FIG. 4E graphically illustrates the absolute cell number (panel a) and frequency (panel b), obtained at 6 and 24 hours after surgery, of leukocyte sub-populations in the peritoneal cavity of WT mice submitted to CLP to induce lethal-grade sepsis, and treated, or not-treated, with recombinant mouse CD5L administered IP.

FIG. 4F graphically illustrates, using geometric mean statistics, the mean fluorescence intensity (MFI) of the Ly6G and CD11b markers expressed by neutrophils, collected at 6 and 24 hours after surgery, from the peritoneal cavity of WT mice submitted to CLP to induce lethal-grade sepsis, and treated, or not-treated, with recombinant mouse CD5L administered IP.

FIG. 4G graphically illustrates the quantification of cytokines by ELISA assay in samples of the peritoneal cavity (panels a and b) and blood serum (panels c and d), collected at 6 and 24 hours after surgery, from WT mice submitted to CLP to induce lethal-grade sepsis, and treated, or not-treated, with recombinant mouse CD5L administered IP.

FIG. 5A graphically illustrates the quantitation by ELISA, at the indicated time-points, of circulating amounts of recombinant mouse CD5L in CD5L-KO mice that were IV injected with 2.5 mg/Kg of the protein.

FIG. 5B schematizes the protocol of CLP-induced sepsis and treatment with recombinant mouse CD5L administered IV in C57BL/6 WT mice, and the analysis of the immune response.

FIG. 5C graphically illustrates the absolute cell number (panel a) and frequency (panel b), obtained at 6 and 24 hours after surgery, of leukocyte sub-populations in the peritoneal cavity of WT mice submitted to CLP to induce lethal-grade sepsis, and treated, or not-treated, with recombinant mouse CD5L administered IV.

FIG. 5D graphically illustrates, using geometric mean statistics, the mean fluorescence intensity (MFI) of the Ly6G and CD11b markers expressed by neutrophils, collected at 6 and 24 hours after surgery, from the peritoneal cavity of WT mice submitted to CLP to induce lethal-grade sepsis, and treated, or not-treated, with recombinant mouse CD5L administered IV.

FIG. 5E graphically illustrates the CFU counts of bacteria, obtained at 6 and 24 hours after surgery, from the peritoneal cavity (panel a) and blood (panel b), grown in aerobic or anaerobic conditions, or from lung (panel c), liver (panel d) and kidney (panel e), grown in aerobiosis, of WT mice submitted to CLP to induce lethal-grade sepsis, and treated, or not-treated, with recombinant mouse CD5L administered IV.

FIG. 5F graphically illustrates the quantification of cytokines by ELISA assay in samples of the peritoneal cavity (panels a and b) and blood serum (panels c and d), collected at 6 and 24 hours after surgery, from WT mice submitted to CLP to induce lethal-grade sepsis, and treated, or not-treated, with recombinant mouse CD5L administered IV.

FIG. 5G graphically illustrates the survival curve of C57BL/6 WT mice undergoing CLP-induced lethal sepsis that were treated with two doses of 2.5 mg/Kg of recombinant mouse CD5L administered IV.

FIG. 6A graphically illustrates the quantification of the chemokine CXCL1 by ELISA assay in samples of the peritoneal cavity and blood serum, of WT and CD5L-KO mice submitted to medium-grade CLP.

FIG. 6B graphically illustrates the quantification of the chemokine CXCL1 by ELISA assay in samples of the peritoneal cavity and blood serum, collected at 6 hours after surgery, from C57BL/6 WT mice submitted to CLP to induce lethal-grade sepsis, and treated, or not-treated, with one dose of 2.5 mg/Kg of recombinant mouse CD5L administered IV 3 hours after surgery.

FIG. 7A graphically illustrates the quantification of CD5L by ELISA assay of blood samples, collected at the indicated days after infection, from C57BL/6 WT mice that were infected IP with 5×10⁴ luciferase-expressing blood stream forms of T. brucei GVR35.

FIG. 7B illustrates the parasite distribution, evaluated by bioluminescence imaging using IVIS Lumina LT at the indicated days after infection, in WT and CD5L-KO mice that were infected IP with 5×10⁴ luciferase-expressing blood stream forms of T. brucei GVR35.

FIG. 7C graphically illustrates the whole-body quantification, in average radiance (photons/s/cm2/steradian), of the bioluminescence images, acquired at the indicated days after infection, from WT and CD5L-KO mice that were infected IP with 5×10⁴ luciferase-expressing blood stream forms of T. brucei GVR35.

FIG. 7D graphically illustrates the parasitemia of infected animals, monitored microscopically by tail-blood examination at the indicated days after infection, from WT and CD5L-KO mice that were infected IP with 5×10⁴ luciferase-expressing blood stream forms of T. brucei GVR35.

FIG. 7E graphically illustrates the survival percentage along time of WT and CD5L-KO mice infected IP with 5×10⁴ luciferase-expressing blood stream forms of T. brucei GVR35.

### DESCRIPTION OF EMBODIMENTS

In a first aspect, the present invention relates to a recombinant human CD5L protein, which is set forth in SEQ ID No. 3. The recombinant human CD5L protein comprises the sequence between amino acids Ser20 and Gly347 of human CD5L fused to an 8-His tag sequence.

The invention further discloses active fragments or peptides derived from the recombinant human CD5L protein, which are set forth in SEQ ID Nos. 4 to 6 and SEQ ID Nos. 7 to 9, respectively.

More specifically, the active fragments comprise amino acids Ser20 to Pro127 of human CD5L or amino acids Ser132 to Pro241 of human CD5L or amino acids Asp240 to Gly347 of human CD5L, fused to an 8-His tag sequence.

The peptides are 11 mer peptides within SRCR domain 1 of human CD5L (amino acids Gly35 to Trp45) or 11 mer peptides within SRCR domain 2 of human CD5L (amino acids Gly149 to Trp159) or 11 mer peptide within SRCR domain 3 of human CD5L (amino acids Gly255 to Trp265).

The recombinant protein and its active fragments are produced from any microbial, mammalian or human sources and can be prepared by methods well known in the art, including translation and expression in a suitable host cell from the nucleic acid encoding the protein of interest.

In a preferred embodiment of the invention, the method of preparation of the recombinant protein and the active fragments thereof uses the protein-expression system of any microbial or mammalian source, preferably Pichia pastoris or HEK293T cells, wherein the recombinant human CD5L protein or an active fragment derived thereof is incorporated into a gene construct or into expression vectors directed to the transfection and expression of the specific polynucleotides.

The peptides derived from the recombinant human CD5L protein are produced synthetically, using methods that will be apparent for those skilled in the art, such as the ones carried out in the biotechnology industry, supposedly under GMP conditions.

In addition, the one or more nucleic acids that code for the mature full-length human CD5L protein or its active fragments, are incorporated into a gene construct or into expression vectors directed to the transfection and expression of oligonucleotides specific for the protein of interest.

In a second aspect of the invention, it is disclosed a pharmaceutical composition comprising:

a therapeutically effective amount of an active principle selected from the group consisting of a recombinant human CD5L protein or one or more active fragments or one or more peptides derived thereof, or one or more nucleic acids encoding full-length human CD5L or active fragments thereof and a suitable excipient, diluent or carrier, wherein the suitable excipient, diluent or carrier is selected from the group consisting of a stabilizing agent or combinations thereof, a surfactant or combinations thereof, a buffering agent or combinations thereof, and an antioxidant or combinations thereof.

More specifically, the pharmaceutical composition comprises:
from 0.1 to 5.0% (by weight) of a recombinant human CD5L protein, or one or more active fragments or peptides derived thereof, or one or more nucleic acids encoding full-length human CD5L or active fragments thereof,
from 2 to 10% (by weight) of a stabilizing agent or a combination thereof;
from 0.05 to 0.1% (by weight) of a surfactant or a combination thereof;
from 0.2% to 0.5% (by weight) of a buffering agent or a combination thereof; and
from 0.01 to 0.04% (by weight) of an antioxidant or a combination thereof.

In a preferred embodiment of the present invention, the human CD5L protein is present in the composition in a recombinant form, as set forth in SEQ ID No. 3.

In another embodiment of the present invention, the active fragments derived from the recombinant human CD5L protein which are present in the composition are set forth in SEQ ID Nos. 4 to 6 and the peptides derived from the recombinant human CD5L protein which are present in the composition are set forth in SEQ ID Nos. 7 to 9.

In a preferred embodiment of the present invention, the stabilizing agent is selected from one or more of the group consisting of di-saccharides such as sucrose or trehalose, sugar alcohols such as mannitol, amino acids such as L-arginine or L-glycine or combinations thereof. In a preferred embodiment of the invention, the stabilizing agent is trehalose.

In a preferred embodiment of the present invention, the surfactant is selected from one or more of the group consisting of polysorbates, such as polysorbate 20 or polysorbate 80, polymers such as polyethylene glycol or combinations thereof. In a preferred embodiment of the invention, the surfactant is polysorbate 80.

In a preferred embodiment of the present invention, the buffering agent is selected from one or more of the group consisting of citrates, phosphates, succinates or combinations thereof. In a preferred embodiment of the invention, the buffering agent is a combination of disodium hydrogen phosphate and sodium dihydrogen phosphate.

In a preferred embodiment of the present invention, the antioxidant is selected from one or more of the group consisting of L-glutathione, L-cysteine, L-methionine or combinations thereof. In a preferred embodiment of the invention, the antioxidant is L-glutathione.

In a third aspect, the present invention describes the use of a pharmaceutical composition comprising a recombinant human CD5L protein, or one or more active fragments or peptides derived thereof, or one or more nucleic acids encoding full-length human CD5L or active fragments thereof, to treat pathological conditions that comprise an intense inflammatory response, acute infections, sepsis or a cytokine storm.

In a preferred embodiment of the present invention, these pathological conditions are selected from one or more of the group consisting of cardiovascular diseases, infectious diseases, parasite diseases, atherosclerosis, type 2 diabetes, rheumatoid arthritis, cancer or immunotherapy, hepatitis due to viral infection or alcohol, acute lung respiratory distress syndrome, cystic fibrosis, chronic obstructive pulmonary disease (COPD), asthma, acute dermatitis, severe acute respiratory syndrome (SARS) and Coronavirus diseases.

As formulated, the pharmaceutical composition of the present invention is compatible with one or more routes of administration of selected from the group consisting of epicutaneous, subcutaneous, intramuscular and intravenous administrations. In a preferred embodiment of the invention, the pharmaceutical composition of the present invention is administered via intravenous route.

In a preferred embodiment of the present invention, the pharmaceutical composition is in a liquid form, or in a solid form for dilution in water and presents a dosage which contains from 2.5 to 5.0 mg per Kg of body mass of a recombinant human CD5L protein, or one or more active fragments or peptides derived thereof, or one or more nucleic acids encoding full-length human CD5L or active fragments thereof.

The administration of the pharmaceutical composition must be performed between 3 to 6 hours to human or animal patients during an exacerbated life-threatening inflammatory syndrome, wherein the blood levels of C-reactive protein (CRP) in the patient reach a value of 50% above normal and the blood oxygen saturation levels drop below 80%.

The invention is further described in the following examples, which do not limit the scope of the invention described in the claims in any way.

### EXAMPLES

Unless otherwise indicated, all technical and scientific terms used in this document have the same meaning as commonly understood by someone skilled in the art to which this invention belongs.

Methods and materials are described in this document for use in the present invention; other suitable methods and materials known in the art can also be used. The materials, methods and examples are illustrative only and are not intended to be limiting.

### Materials and Methods Used:

### Animals

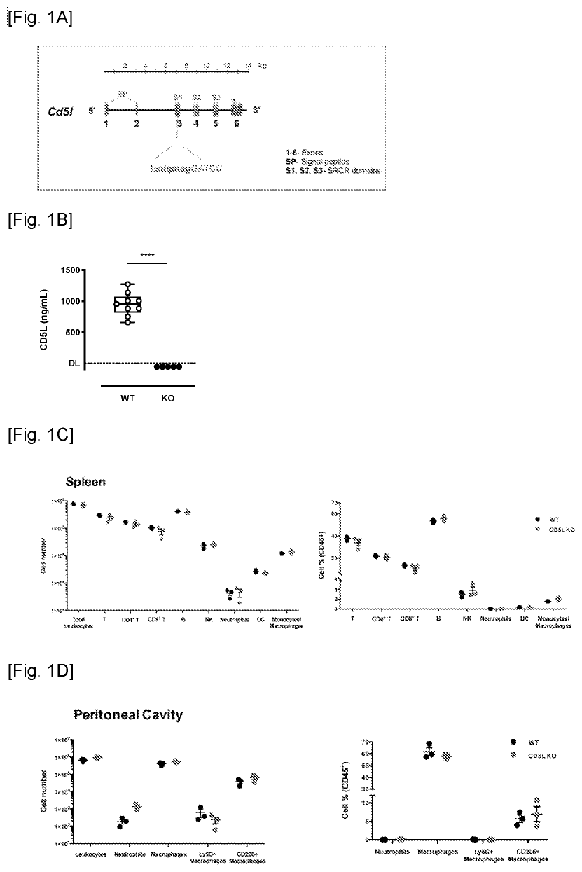

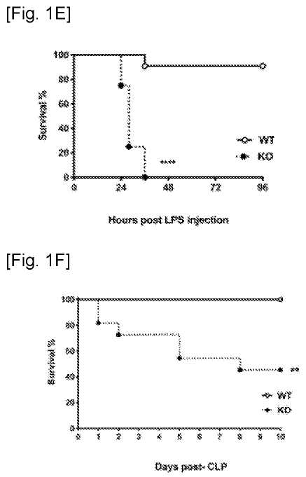

Knockout (KO) mice for CD5L were generated using CRISPR/Cas9 engineering, according to a method already known in the prior art (FIG. 1A).

In summary, the Cas9 mRNA and sgRNAs targeting exon 3 of the Cd5/gene were produced by in vitro transcription. Homologous recombination was promoted by a single-stranded DNA (ss) oligonucleotide comprising 120 nucleotides containing sequences of 60 nucleotides on each side of the deletion separated by an EcoRI site and three tandem stop codons. Cas9 mRNA (10 ng/ml), sgRNA1 and sgRNA2 (10 ng/ml each) and ssDNA oligonucleotide (10 ng/ml) were injected into the C57BL/6 fertilized oocyte pronucleus using standard procedures. Deletions were evaluated by PCR of the genomic DNA of the tail and then confirmed by direct sequencing.

The experiments were carried out on C57BL/6 mice with 8 to 12 weeks of age, following Portuguese and European legislation (namely, Portaria 1005/92 and Directive 2010/63/EU) on accommodation, breeding and animal welfare.

The project was analyzed and approved by the Ethics Committee of the Institute of Research and Innovation in Health (i3S) of the University of Porto and by the General Directorate of Food and Veterinary of the Portuguese National Entity (license reference: 009951).

### Cloning, Production and Purification of Mouse and Human CD5L Protein

Cloning, production and purification of the mouse and human forms of the CD5L protein were performed. The transfection and expression of the specific polynucleotides can be performed by using the protein-expression system of any microbial or mammalian source.

For efficient secretory expression, the murine and human CD5L cDNA fragments were placed downstream of the coding sequence for the human CD14 protein signal peptide (Met1-Val17). Briefly, mouse CD5L (mCD5L) mRNA was amplified from murine cDNA extracts and inserted into mammalian expression vector pCD14SP-8HIS_Zeo (derived from pcDNA3.1/Zeo (+) via NheI and BamHI restriction sites. The resulting translated sequence comprises a C-terminally fused 8× histidine marker.

The coding sequence for human CD5L protein (hCD5L) was cloned by PCR amplification from human spleen cDNA using the primers set forth in SEQ IDs 1 and 2, digested with NheI and BamHI restriction endonucleases and ligated into pCD14SP-8HIS_Zeo. The recombinant human CD5L protein comprises the sequence between amino acids Ser20 and Gly347 of human CD5L fused to an 8-His tag sequence, as set forth in SEQ ID No. 3.

The resulting plasmids pCD14SP-mCD5L-8HIS_Zeo and pCD14SP-hCD5L-8HIS_Zeo were transfected into mammalian cells, preferably HEK293T cells, and the cells were selected by supplementation with the appropriate selection antibiotic, preferably of 150 μg/ml of zeocin. The cells expressing the human and mouse CD5L proteins in a stable manner were cultured in a rolling-bottle incubator.

Culture supernatants were collected and subjected to ultrafiltration (Sartocon Slice Cassette of 5 KDa) to replace the culture medium with 50 mM Tris and 200 mM NaCl pH 7.8 (Tris/NaCl). The samples were purified by immobilized metal affinity chromatography (IMAC) using Ni-NTA-Agarose-Resin (Genaxxon Bioscience). The resin was washed with Tris/NaCl/50 mM imidazole followed by elution with Tris/NaCl/350 mM imidazole.

Fractions containing human or mouse recombinant CD5L were identified using SDS-PAGE, pooled and diluted with Tris/NaCl (1:20). For further purification, the samples were loaded onto Q-Sepharose FF (GE Healthcare), washed with Tris/NaCl and eluted with a linear gradient to 1 M NaCl. After dialysis and additional enrichment in centrifugal concentrators, the protein concentration was determined by photometric measurement (A280 nm) and silver-stained SDS-PAGE was performed to confirm homogeneity greater than 95%.

The endotoxicity of the 0.22 μm filtered mCD5L solution was assessed to be less than 1 EU/μg of protein by the chromogenic endpoint assay of the amebocyte lysate of Limulus sp. (Associates of Cape Cod Europe GmbH). The human and mouse recombinant CD5L protein preparations were aliquoted and lyophilized for convenient storage and use. The sequence of the human CD5L protein can be verified in SEQ ID No. 3.

Optionally, a fermentative production of recombinant hCD5L in Pichia pastoris protein-expression system is used, wherein the fermentation is performed in 1, 2, 20 or 140 L fermenters.

The temperature is set at 30° C., agitation at 500 rpm and aeration rate at 1 vvm. The pH is adjusted to pH 5.0 with 25% ammonium hydroxide and the fermenter is inoculated with 10% of the initial fermentation volume of a culture grown in minimal glycerol medium.

A batch culture is grown until the glycerol is completely consumed, in which aeration and agitation are increased to 2 vvm and 1000 rpm, respectively. A glycerol fed-batch phase is initiated by feeding 50% glycerol containing 12 ml/L trace salts, at a rate of 18 ml/h per liter of initial fermentation volume. The pH is maintained at 5.0 and the glycerol feed is discontinued when the cell wet-weight reaches approximately 180 g/l.

After complete consumption of the glycerol, additional casamino acids are optionally added to 1% of the initial fermenter volume and the culture is induced by initiating a 100% methanol feed containing 12 ml/l trace salts. The feed rate is initially set at 3 ml/h per liter initial fermenter volume, and gradually increased to maximally 9 ml/h per liter initial fermenter volume.

Except when specifically indicated otherwise, the recombinant mCD5L protein was used in the experimental methods performed in mice.

### Immunofluorescence

Mouse peritoneal cells were adhered to glass slides coated with poly-L-lysine for 30 minutes at 37° C. followed by fixation with 4% PFA.

Staining the surface with an anti-F4/80 anti-mouse antibody was performed followed by a secondary anti-mouse antibody coupled to AlexaFluor 594.

Intracellular staining with polyclonal goat anti-CD5L antibody was performed followed by a secondary donkey goat antibody coupled to AlexaFluor 488 after permeabilization with Triton X100.

Dapi was used as a nuclear counter stain and the cells were visualized under a Leica SP5 confocal microscope.

### ELISA

The quantification of mCD5L was performed using the set of ELISA pairs for mouse CD5L (SinoBiological) according to the manufacturer's instructions.

### Flow Cytometry and Multiplex Assays

For the analysis of cell populations, cells were recovered, washed with flow staining medium (FSM, PBS containing 2% FBS) and 1×10⁶ cells were stained with fluorophore-conjugated antibodies against surface markers.

When necessary, cells were fixed with 2% PFA and permeabilized with 0.1% saponin in FSM before intracellular staining with anti-mCD5L. Cells were analyzed on a FACS Canto II flow cytometer using FACS Diva.

Mouse cytokine (LEGENDplex™ Mouse Inflammation Panel, Biolegend) and chemokine (LEGENDplex™ Mouse Proinflammatory Chemokine Panel, Biolegend) were quantified in mouse sera or peritoneal fluid following the provided instructions. Samples were acquired in a Accuri C6 (BD) flow cytometer.

Analysis was performed in FlowJo software.

### Septic Shock Induced by Lipopolysaccharide (LPS)

To induce septic shock, mice were injected intraperitoneally (IP) with lipopolysaccharides (LPS) from Escherichia coli O111: B4 (Sigma-Aldrich) at doses of 5 mg/Kg body mass (sub-lethal model) or 10 mg/Kg (lethal model). Mice were monitored twice a day.

### Cecum Ligation and Puncture Model

For sepsis induction by cecum ligation and puncture (CLP), the mice underwent surgery under anesthesia with isoflurane, exposure and cecum ligation (corresponding to approximately half the distance between the distal pole and the cecum base for sepsis of medium degree, or 75% of the distance for high degree sepsis) and direct puncture with a 21G needle.

After suturing, mice were injected subcutaneously (sc) with 0.9% NaCl. Buprenorphine (0.08 mg/Kg) was administered subcutaneously every 12 hours up to 48 hours after surgery. Mice were monitored twice a day.

### Bacterial Counts

For the quantification of bacteria in the indicated organs, 10-fold serial dilutions of cell suspensions were seeded on Brain Heart Infusion (BHI) agar plates and cultured under aerobic or anaerobic conditions using the AnaeroJar and AnaeroGen (Oxoid, Thermo Scientific) sachets for 18 hours at 37° C.

### Histology

Sections of pulmonary, hepatic and renal tissue were fixed in neutral buffered formaldehyde prior to paraffin incorporation. 4 μm sections were stained with hematoxylin and eosin. The lung, liver and kidney pathology scores were assigned by an independent pathologist in a double-blind analysis, in which:
0—no sign of inflammation,
1—minimal inflammatory signs,
2—mild inflammation,
3—moderate to severe inflammation.

### Experiments Performed:

Experiment 1—CD5L-KO Mice are More Susceptible to Sepsis than WT Mice

In order to explore the role of CD5L in sepsis and other disease models, mice deficient for this gene/protein were generated by CRISPR/Cas9 engineering, targeting exon 3 of the CD5L gene in C57BL/6 mice with the insertion of three in-frame stop codons and a frame shift (FIG. 1A).

The efficient deletion of CD5L in CD5L-KO mice was confirmed by ELISA, which revealed the presence of high levels of the protein in the blood of WT mice and the complete absence of the protein in CD5L-KO mice (FIG. 1B). The main source of CD5L are tissue macrophages, and whereas peritoneal F4/80⁺ macrophages of WT mice expressed the protein, as detected by immunofluorescence, equivalent cells from CD5L-KO mice lacked the expression of CD5L (Panel “CD5L” in FIG. 1C).

The analysis of basal cell populations in the spleen (FIG. 1D) and in the peritoneal cavity (FIG. 1E) did not reveal any significant differences between WT and CD5L-KO mice in absolute numbers or frequencies of the different types of cell in the resting state.

The susceptibility of CD5L-KO mice to endotoxin challenge was investigated in a model of sterile sepsis, with the IP administration of LPS. After the injection of a sublethal dose of LPS (5 mg/Kg), all CD5L-KO mice died between 24 and 36 hours after the challenge, in contrast with the survival of 90% of the WT mice (FIG. 1F).

Both groups of mice were also subjected to the gold standard sepsis model induced by a polymicrobial infection by means of ligation and puncture of the cecum. While 100% of the WT mice survived after inducing medium-grade sepsis, 50% of the KO mice succumbed (FIG. 1G), further confirming the increased susceptibility in sepsis settings.

### Experiment 2—CD5L-KO Mice Show Impaired Immune Response to Sepsis

In order to better understand the increased susceptibility of CD5L-KO mice to sepsis, different aspects of the immune response induced in WT and CD5L-KO mice were characterized at 6 and 24 hours after CLP.

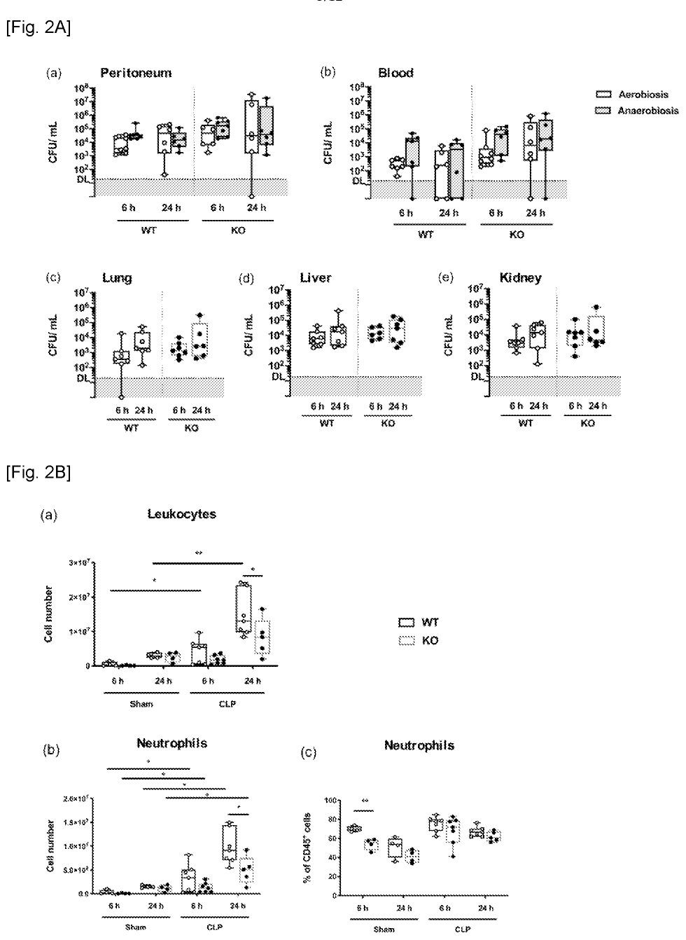

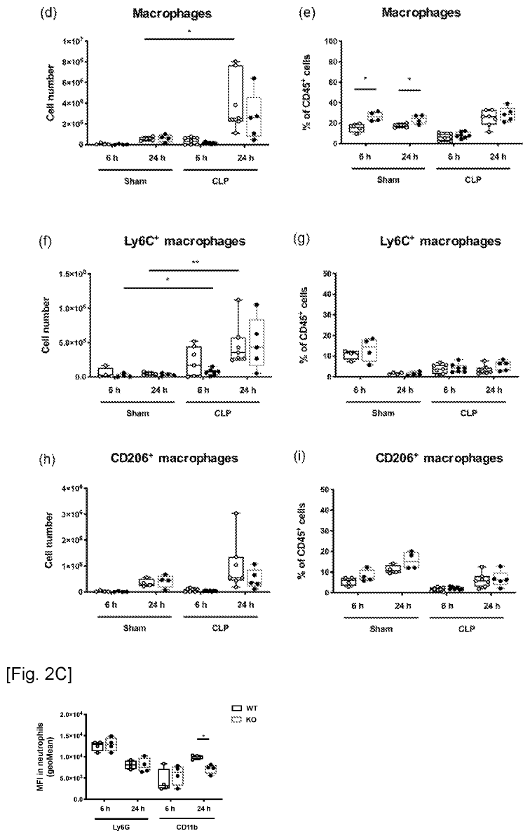

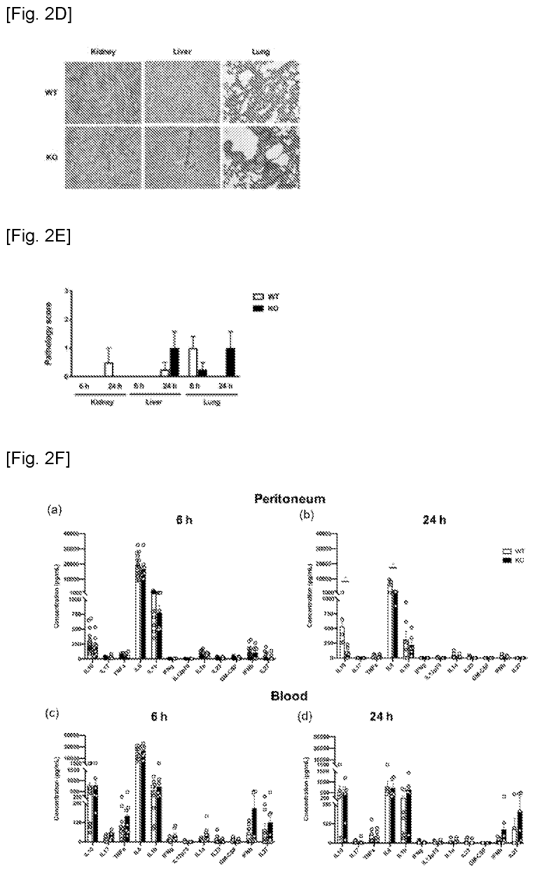

Counts of colony-forming units (CFU) of both aerobic and anaerobic bacteria showed that CD5L-KO mice presented modest increases in the levels of bacteria in the peritoneal cavity already at 6 hours post-surgery, compared with WT mice, and blood bacteremia at 24 hours (FIG. 2A). However, no differences in CFUs between the two groups were detected in the lungs, liver or kidneys at 6 or 24 hours after CLP (FIG. 2A, lower panels).

After surgery, cell populations in the peritoneal cavity changed dramatically with an increase in myeloid cells, particularly neutrophils that infiltrate this space quickly after injury or infection. Mice undergoing CLP showed a more pronounced increase in infiltrating cells in the peritoneal cavity than animals undergoing a sham procedure, as expected, due to the presence of bacteria being released from the cecum (FIG. 2B, panel “Leukocytes”). Importantly, CD5L-KO mice have a significantly lower number of infiltrating cells, mostly due to a reduced influx of neutrophils, evident within 24 hours after surgery, and also a slight reduction in the numbers of macrophages, compared with WT mice (FIG. 2B, panels “Neutrophils”).

Given that CD5L has been previously shown to be involved in the acquisition of the M2 phenotype by macrophages, it was further assessed whether there was an imbalance between the M1 and M2 phenotypes in the peritoneal macrophages using the classical markers Ly6C and CD206, respectively. However, no significant differences were observed between the WT and CD5L-KO mice (FIG. 2B, panels “Ly6C⁺ macrophages” and “CD206⁺ macrophages”).

In addition to impaired neutrophil recruitment, these cells show a different phenotype in CD5L-KO mice, marked by reduced surface expression of α-integrin CD11b+, consistent with a less activated state, while Ly6G expression remains unchanged (FIG. 2C).

A more systemic analysis indicated that 24 hours after surgery the CD5L-KO mice showed increased the signs of pathology in the lungs and liver compared with the WT mice (FIG. 2D, immunohistochemistry; FIG. 2E, quantification of pathology scores).

A multiplex analysis of important inflammation-related cytokines in the peritoneal fluids and blood revealed that CD5L-KO mice produced significantly lower amounts of IL-6 and IL-10 than WT mice, but only in the peritoneal cavity and at 24 h after CLP (FIG. 2F).

### Experiment 3—CD5L Protein Levels Change Upon Infection and CD5L is Trafficked to the Site of Infection

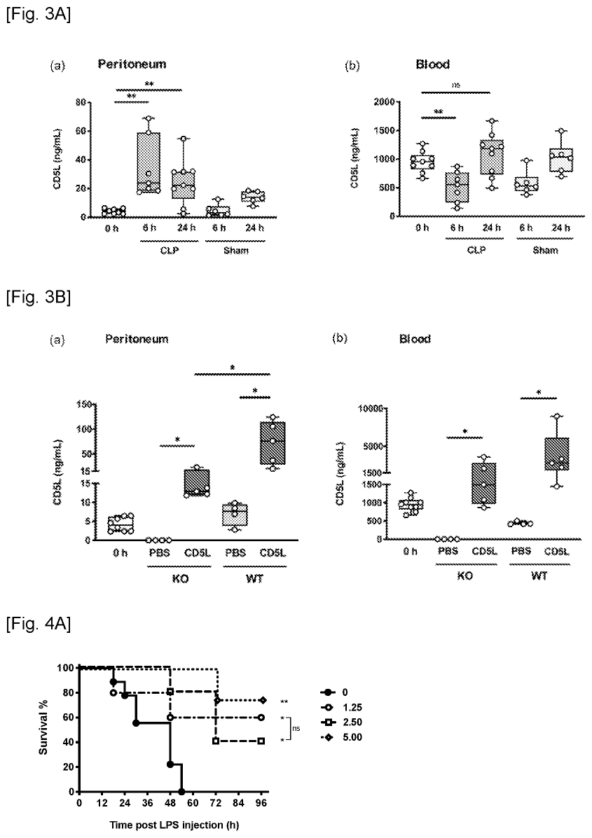

To understand the role of CD5L in the context of sepsis, this protein was quantified in the peritoneal cavity after CLP. CD5L levels increased significantly in the peritoneal cavity after aggression as early as 6 hours after challenge, decreasing at 24 hours, but remaining significantly higher than in healthy controls (0 h) (FIG. 3A, left panel). On the other hand, circulating levels of CD5L decreased 6 h after CLP, returning to normal at 24 hours (FIG. 3A, right panel).

To investigate whether the decrease in circulating levels of CD5L was a consequence of the mobilization of the protein to the site of the aggression, recombinant mCD5L was injected intravenously (IV) at 2.5 mg/Kg into CD5L-KO mice 3 h after CLP, and peritoneal fluid and blood were recovered 1 hour later to quantify the total amount of CD5L by ELISA. One hour after IV administration (4 h after CLP), the amount of CD5L in the blood of the CD5L-KO mice was quantified at ˜1-2 g/ml (FIG. 3B, right panel) and, importantly, CD5L was clearly detected in the peritoneal fluid (FIG. 3B, left panel), confirming a direct trafficking between the blood and the infection site. The same procedure performed on WT mice resulted in an even bigger increase of rCD5L in the peritoneal cavity and in blood (FIG. 3B, left and right panels).

### Experiment 4—CD5L IP Administration Prevents Lethality Upon Sepsis Induction

Since the absence of CD5L in CD5L-KO mice increases the lethality in mouse models with sepsis and there is a significant increase in the said protein in C57BL/6 WT animals after CLP infection, it was investigated whether the exogenous addition of the recombinant mCD5L protein would improve the outcome of sepsis induction.

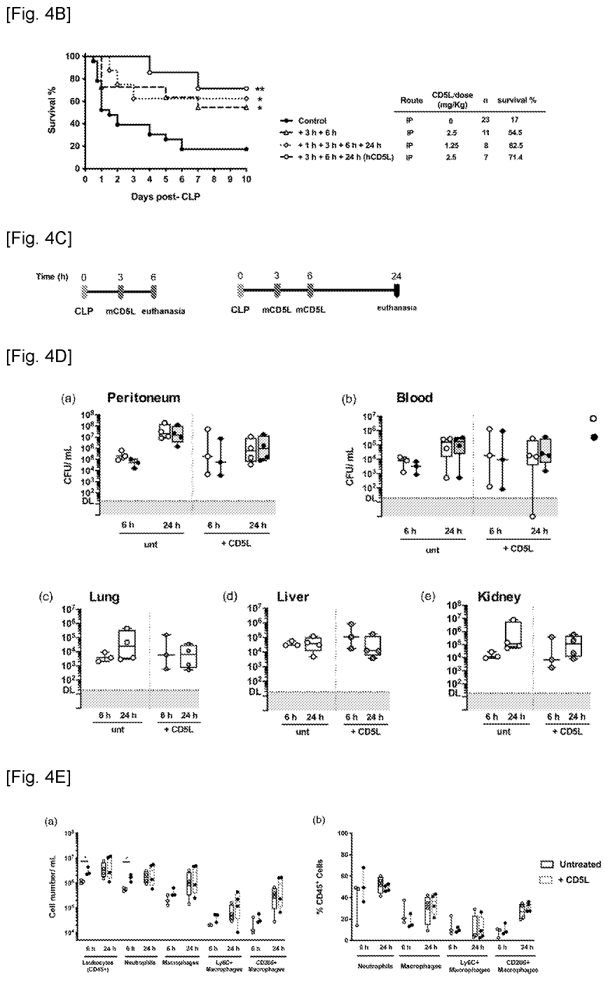

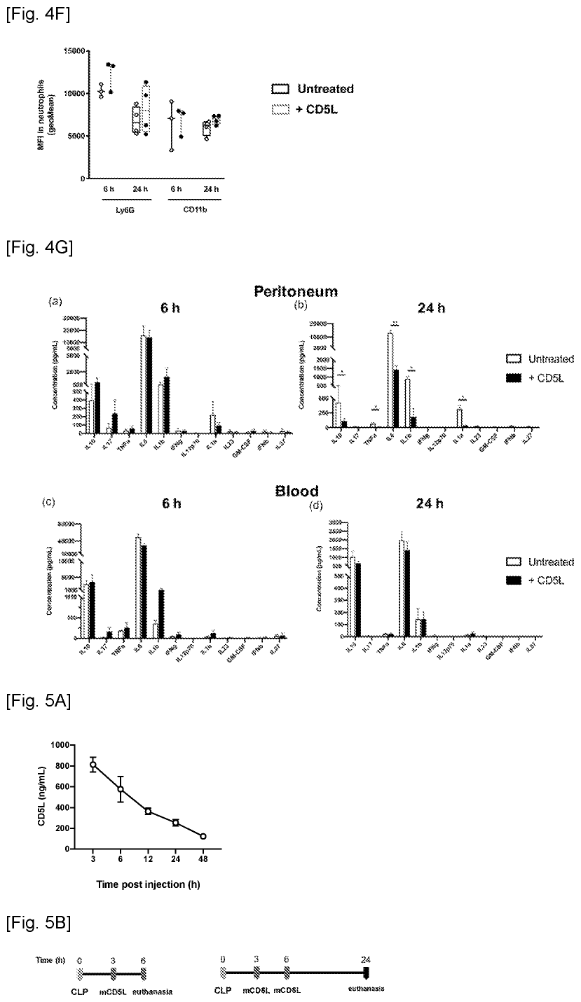

C57BL/6 WT mice received a lethal dose (10 mg/Kg) of LPS, and 3 groups of mice received doses of 1.25, 2.5 or 5 mg/Kg of recombinant mCD5L, given IP 3 hours after the LPS challenge, while a fourth group received PBS only (untreated). Whereas untreated mice all died within 2.5 days (FIG. 4A, solid line-black circles), mice having received rCD5L at different doses had a survival rate of over 40% four days after the LPS challenge (FIG. 4A, dashed lines).

WT mice were also subjected to CLP to induce high-grade sepsis following which a group received sequential doses, injected IP, of 2.5 mg/Kg of recombinant mCD5L after 3 and 6 hours, while the control group received PBS alone at the same time points. Survival was monitored for 10 days, and whereas untreated mice had a low survival rate (17%) (FIG. 4B, solid line-black circles), the percentage of survival of the animals treated with mCD5L was 55% (FIG. 4B, dashed line-white triangles). Distributing the dosage of recombinant mCD5L through four doses of 1.25 mg/Kg administered at 1, 3, 6 and 24 hours resulted in a similar survival rate (63%) (FIG. 4B, dotted line-white prisms).

To evaluate the possible translation of the treatment to human medicine, recombinant human CD5L was produced and its therapeutic effect was tested in the same mouse model. IP injection of 2.5 mg/Kg of recombinant hCD5L after 3, 6 and 24 hours of CLP resulted in a similar survival rate as the recombinant mouse protein (71%) (FIG. 4B, solid line-white circles).

To understand the molecular mechanisms involved in CD5L-mediated protection given IP, the immune responses were analyzed: i) at 6 hours after CLP in mice that had been treated with a single dose of 2.5 mg/Kg of recombinant mouse CD5L given IP 3 hours post-CLP (FIG. 4C, left); and ii) at 24 hours in mice that had received two IP doses of 2.5 mg/Kg of recombinant mCD5L, at 3 and 6 hours after CLP (FIG. 4C, right).

Bacterial counts were measured in the peritoneal cavity, blood, lungs, liver and kidneys, and although the CFUs showed a tendency to be lower at 24 hours after CLP in the treated mice, the differences were not significant (FIG. 4D).

A detailed analysis of the infiltrating cells in the peritoneal cavity showed that recombinant mCD5L IP treatment resulted in a rapid increase in the number of neutrophils recruited at 6 hours after CLP (3 hours after first dose of recombinant mCD5L) (FIG. 4E, left panel). However, the activation phenotype of the recruited neutrophils was not different comparing treated with untreated mice (FIG. 4F).

At 24 hours after CLP and with 2 doses of recombinant mCD5L, there was a substantially reduction of the amounts of inflammatory mediators such as TNF-α, IL-6, IL-1a and IL-1B and the anti-inflammatory cytokine IL-10 in the peritoneum of treated animals, compared with the untreated group (FIG. 4G, top-right panel). However, no major differences in cytokine production were detected in the blood of both groups of mice during the treatment (FIG. 4G, lower panels).

### Experiment 5—IV Therapeutic Administration of Recombinant Mouse CD5L Promotes Bacterial Clearance, Reduces Inflammation, and Significantly Increases Survival Upon Polymicrobial Infection and Sepsis

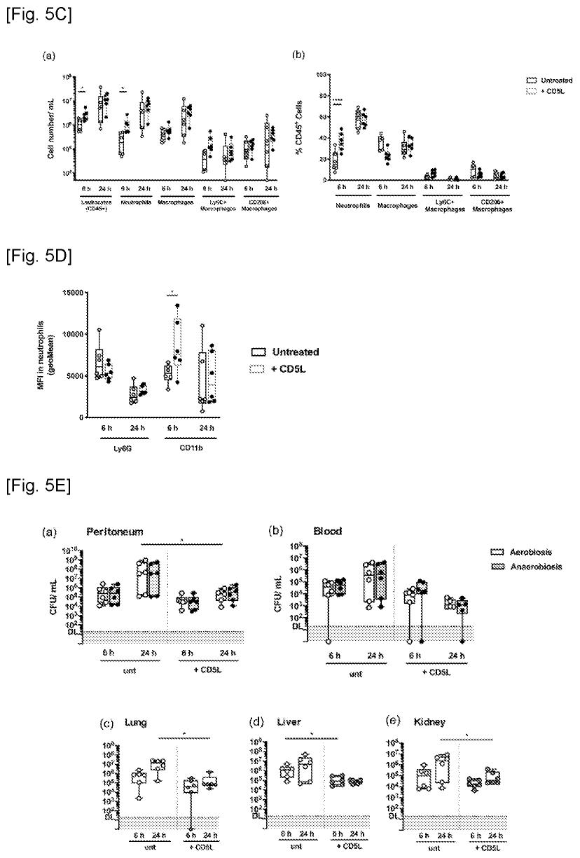

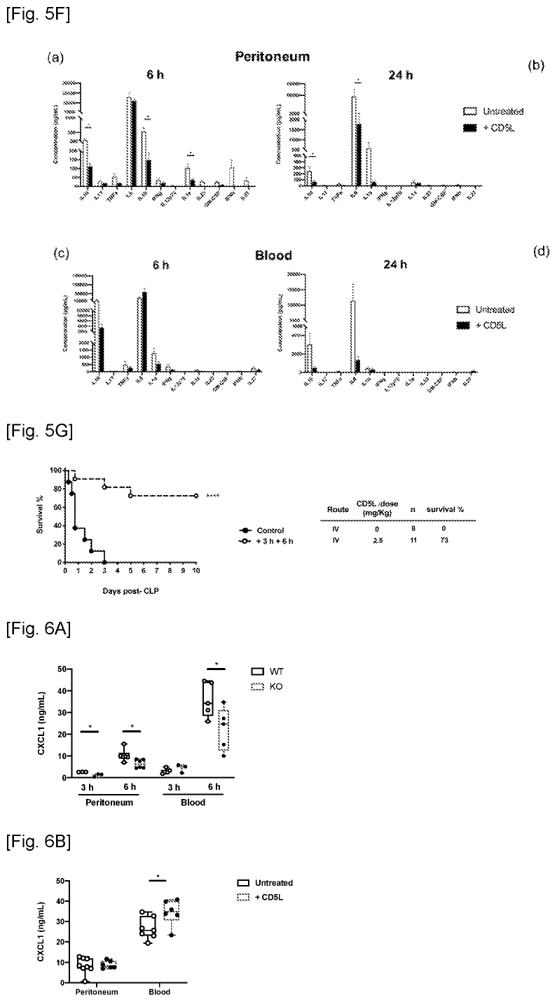

The IV administration of recombinant mCD5L was assessed, as this represents a preferred administration route in human therapy. First, the half-life of CD5L in the blood was checked after intravenous injection of 2.5 mg/Kg in CD5L-KO mice, and it was found to be approximately 10 hours (FIG. 5A).

To understand the molecular mechanisms involved in CD5L-mediated protection given IV, the immune responses were analyzed in C57BL/6 WT mice: i) at 6 hours after CLP in mice that had been treated with a single dose of 2.5 mg/Kg of recombinant mouse CD5L given IV 3 hours post-CLP (FIG. 5B, left).; and ii) at 24 hours in mice that had received two IV doses of 2.5 mg/Kg of recombinant mCD5L, at 3 and 6 hours after CLP (FIG. 5B, right).

IV treatment with recombinant mCD5L was characterized by a fast recruitment of neutrophils, both in number (FIG. 5C, left panel) as well in percentage, to the site of infection (FIG. 5C, right panel). To note, the recruited neutrophils displayed an activated phenotype (FIG. 5D).

As result of the fast and efficient response following IV administration of recombinant mCD5L, there was a clear reduction in the aerobic and anaerobic bacterial loads, both locally (FIG. 5E, top-left panel) as well as systemically in the organs analyzed, comparing CD5L-treated with untreated mice (FIG. 5E, bottom panels).

The reduction of some inflammatory mediators in the peritoneal cavity after recombinant mCD5L IV administration was much faster than that seen for IP-treated animals, as one single dose of rCD5L reduced the levels of the inflammatory cytokines IL-1a and IL-1B already 3 hours after treatment (6 hours after CLP) (FIG. 5F, top-left panel). The anti-inflammatory mediator IL-10 was also decreased faster upon IV than IP treatment (FIG. 5F, top panels). No significant differences were observed in the cytokine profile obtained in the blood of IV-treated mice, when compared with untreated animals (FIG. 5F, bottom panels).

After the characterization of the biological and immunological parameters following the IV administration of recombinant mCD5L, the efficacy of such therapy to fight CLP-induced sepsis was evaluated. C57BL/6 WT mice were submitted to the lethal CLP procedure, and a group of mice received two doses of 2.5 mg/Kg of recombinant mCD5L injected IV at 3 and 6 hours after CLP, while another group received vehicle alone at the same time points. This therapeutic procedure was very effective as the treated animals showed a survival rate of 73% (FIG. 5G, dashed line white circles), compared with no survival (0%) for the mice receiving no treatment (FIG. 5G, solid line black circles).

Experiment 6—Recombinant Mouse CD5L Administration is Associated with Increased CXCL1 Levels Responsible for Accentuated Neutrophil Chemotaxis

Given that that the deficiency in CD5L in the CD5L-KO mice resulted in decreased immune cell recruitment upon CLP, and that by opposition the therapeutic administration of recombinant mCD5L increased neutrophil numbers in the peritoneum, the involvement of CD5L in the mechanisms of neutrophil recruitment were analyzed.

The fluids from the peritoneal cavity and blood of C57BL/6 WT and CD5L-KO mice recovered 3 and 6 hours after CLP, and from mice treated IV with recombinant mCD5L at 3 hours after CLP and euthanized at 6 hours were analyzed using an inflammatory chemokine array.

CXCL1, a chemokine connoted with neutrophil chemotaxis, was consistently decreased in the peritoneal cavity and blood of CD5L-KO mice, when compared with WT mice, at 3 and 6 hours after CLP (FIG. 6A).

Conversely, mice that were IV-treated with recombinant mCD5L had significantly higher blood levels of CXCL1 at 6 hours after CLP than those untreated (FIG. 6B).

Experiment 7—CD5L-KO Mice are More Susceptible to Infection by Trypanosoma brucei than WT Mice

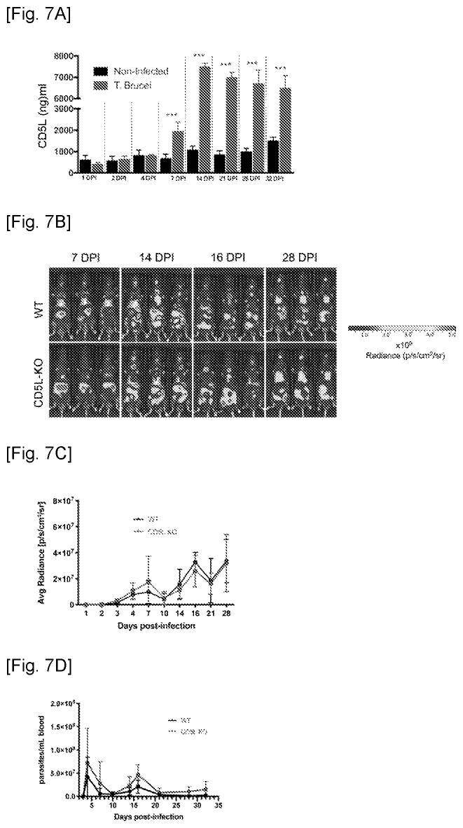

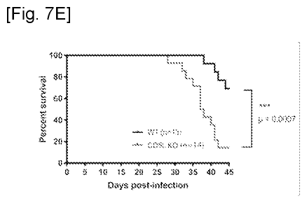

C57BL/6 WT mice infected IP with 5×10⁴ luciferase-expressing blood stream forms of T. brucei GVR35 showed increased circulating levels of CD5L, as determined by ELISA, from day 7 of infection until the end of the experiment, compared with non-infected mice (FIG. 7A).

The susceptibility of CD5L-KO mice to infection by T. brucei GVR35 was evaluated. C57BL/6 WT and CD5L-KO mice were infected IP with 5×10⁴ luciferase-expressing blood stream forms of T. brucei GVR35, and parasite distribution was evaluated by bioluminescence imaging in different time points after infection using IVIS Lumina LT (FIG. 7B). Quantification of the bioluminescence signal in average radiance (photons/s/cm2/steradian) showed no major differences in parasite load in the different organs (FIG. 7C). No measure of whole-body parasite load was performed after 28 days post-infection, since the animals may not survive anesthesia for the IVIS analysis.

Also, no differences in parasitemia between WT and CD5L-KO mice were observed following microscopically tail-blood examination in different time-points (FIG. 7D).

However, CD5L-KO mice (FIG. 7E, grey line) succumbed much faster to the infection than WT mice (FIG. 7E, black line).

Overall, the results show the possible involvement of the CD5L protein as a facilitating agent for the recruitment of neutrophils.

The subject matter described above is provided as an illustration of the present invention and, therefore, should not be construed to limit it. The terminology employed for the purpose of describing preferred embodiments of the present invention should not be restricted to them.

As used in the description, defined and indefinite articles, in their singular form, are intended for interpretation to also include plural forms, unless the context of the description explicitly indicates otherwise.

Undefined articles “one” should generally be interpreted as “one or more”, unless the meaning of a singular modality is clearly defined in a specific situation.

It will be understood that the terms “understand” and “include”, when used in this description, specify the presence of characteristics, elements, components, steps and related operations, but do not exclude the possibility of other characteristics, elements, components, steps and operations as well contemplated.

As used throughout this patent application, the term “or” is used in an inclusive sense rather than an exclusive sense, unless the exclusive meaning is clearly defined in a specific situation. In this context, a phrase of the type “X uses A or B” should be interpreted as including all relevant inclusive combinations, for example “X uses A”, “X uses B” and “X uses A and B”.

In addition, the articles “a” and “an” as used in this application and the appended claims should generally be construed to mean “one or more” unless specified otherwise or clear from the context to be directed to a singular form.

All changes, provided they do not modify the essential characteristics of the following claims, must be considered within the scope of the protection of the present invention.

Sequence Listing Free Text

SEQ. ID. 1
CGTCGCTAGCGCATCTCCATCTGGAGTGCGGC
SEQ. ID. 2
CAAGGATCCTCCTGAGCAGATGACAGCCAC
SEQ. ID. 3
SPSGVRLVGGLHRCEGRVEVEQKGQWGTVCDDGWDIKDVAVLCRELGCG
AASGTPSGILYEPPAEKEQKVLIQSVSCTGTEDTLAQCEQEEVYDCSHDEDAGA
SCENPESSFSPVPEGVRLADGPGHCKGRVEVKHQNQWYTVCQTGWSLRAAK
VVCRQLGCGRAVLTQKRCNKHAYGRKPIWLSQMSCSGREATLQDCPSGPWG
KNTCNHDEDTWVECEDPFDLRLVGGDNLCSGRLEVLHKGVWGSVCDDNWGE
KEDQVVCKQLGCGKSLSPSFRDRKCYGPGVGRIWLDNVRCSGEEQSLEQCQH
RFWGFHDCTHQEDVAVICSGGSSHHHHHHHH
SEQ. ID. 4
SPSGVRLVGGLHRCEGRVEVEQKGQWGTVCDDGWDIKDVAVLCRELGCG
AASGTPSGILYEPPAEKEQKVLIQSVSCTGTEDTLAQCEQEEVYDCSHDEDAGA
SCENPGSSHHHHHHHH
SEQ. ID. 5
SPVPEGVRLADGPGHCKGRVEVKHQNQWYTVCQTGWSLRAAKVVCRQLG
CGRAVLTQKRCNKHAYGRKPIWLSQMSCSGREATLQDCPSGPWGKNTCNHD
EDTWVECEDPGSSHHHHHHHH
SEQ. ID. 6
DPFDLRLVGGDNLCSGRLEVLHKGVWGSVCDDNWGEKEDQVVCKQLGCG
KSLSPSFRDRKCYGPGVGRIWLDNVRCSGEEQSLEQCQHRFWGFHDCTHQE
DVAVICSGGSSHHHHHHHH
SEQ. ID. 7
GRVEVEQKGQW
SEQ. ID. 8
GRVEVKHQNQW
SEQ. ID. 9
GRLEVLHKGVW
SEQ. ID. 10
gatgctcgatatacgactcactatagggagacccaagctggctagcgtttaaacttaagcttatg
gctctgctattctccttgatccttgccatttgcaccagacctggattcctagcgtctccatctgg
agtgcggctggtggggggcctccaccgctgtgaagggcgggtggaggtggaacagaaaggccagt
ggggcaccgtgtgtggctacggctgggacattaaggacgtggctgtgttgtgccgggagctgggc
tgtggagctgccagcggaacccctagtggtattttgtatgagccaccagcagaaaaagagcaaaa
ggtcctcatccaatcagtcagttgcacaggaacagaagatacattggctcagtgtgagcaagaag
aagtttatgattgttcacatgatgaagatgctggggcatcgtgtgagaacccagagagctctttc
tccccagtcccagagggtgtcaggctggctgacggccctgggcattgcaagggacgcgtggaagt
gaagcaccagaaccagtggtataccgtgtgccagacaggctggagcctccgggccgcaaaggtgg
tgtgccggcagctgggatgtgggagggctgtactgactcaaaaacgctgcaacaagcatgcctat
ggccgaaaacccatctggctgagccagatgtcatgctcaggacgagaagcaacccttcaggattg
cccttctgggccttgggggaagaacacctgcaaccatgatgaagacacgtgggtcgaatgtgaag
atccctttgacttgagactagtaggaggagacaacctctgctctgggcgactggaggtgctgcac
aagggcgtatggggctctgtctgtgatgacaactggggagaaaaggaggaccaggtggtatgcaa
gcaactgggctgtgggaagtccctctctccctccttcagagaccggaaatgctatggccctgggg
ttggccgcatctggctggataatgttcgttgctcaggggaggagcagtccctggagcagtgccag
cacagattttgggggtttcacgactgcacccaccaggaagatgtggctgtcatctgctcaggagg
atcctcccatcaccatcaccatcaccatcactgat

### CITATION LIST

Here follows the list of citations:

### Patent Literature

PTL1: WO2018218231A1
PTL2: WO2017087708A1

### Non Patent Literature

NPL1: Bessa Pereira, C., Bockova, M., Santos, R. F., Santos, A. M., de Araujo, M. M., Oliveira, L., Homola, J., and Carmo, A. M. (2016). The Scavenger Receptor SSc5D Physically Interacts with Bacteria through the SRCR-Containing N-Terminal Domain. Front Immunol 7, 9.
NPL2: Elomaa, O., Kangas, M., Sahlberg, C., Tuukkanen, J., Sormunen, R., Liakka, A., Thesleff, I., Kraal, G., and Tryggvason, K. (1995). Cloning of a novel bacteria-binding receptor structurally related to scavenger receptors and expressed in a subset of macrophages. Cell 80, 603-609.
NPL3: Fabriek, B. O., van Bruggen, R., Deng, D. M., Ligtenberg, A. J., Nazmi, K., Schornagel, K., Vloet, R. P., Dijkstra, C. D., and van den Berg, T. K. (2009). The macrophage scavenger receptor CD163 functions as an innate immune sensor for bacteria. Blood 113, 887-892.
NPL4: Gao, X., Yan, X., Zhang, Q., Yin, Y., and Cao, J. (2019). CD5L contributes to the pathogenesis of methicillin-resistant Staphylococcus aureus-induced pneumonia. Int Immunopharmacol 72, 40-47.
NPL5: Hampton, R. Y., Golenbock, D. T., Penman, M., Krieger, M., and Raetz, C. R. (1991). Recognition and plasma clearance of endotoxin by scavenger receptors. Nature 352, 342-344.
NPL6: Holmskov, U., Mollenhauer, J., Madsen, J., Vitved, L., Gronlund, J., Tornoe, I., Kliem, A., Reid, K. B., Poustka, A., and Skjodt, K. (1999). Cloning of gp-340, a putative opsonin receptor for lung surfactant protein D. Proc Natl Acad Sci USA 96, 10794-10799.
NPL7: Martínez, V. G., Escoda-Ferran, C., Tadeu Simões, I., Arai, S., Orta Mascaró, M., Carreras, E., Martinez-Florensa, M., Yelamos, J., Miyazaki, T., and Lozano, F. (2014). The macrophage soluble receptor AIM/Api6/CD5L displays a broad pathogen recognition spectrum and is involved in early response to microbial aggression. Cell Mol Immunol 11, 343-354.
NPL8: Rudd, K. E., Johnson, S. C., Agesa, K. M., Shackelford, K. A., Tsoi, D., Kievlan, D. R., Colombara, D. V., Ikuta, K. S., Kissoon, N., Finfer, S., et al. (2020). Global, regional, and national sepsis incidence and mortality, 1990-2017: analysis for the Global Burden of Disease Study. Lancet 395, 200-211.
NPL9: Sanjurjo, L., Amezaga, N., Aran, G., Naranjo-Gomez, M., Arias, L., Armengol, C., Borras, F. E., and Sarrias, M. R. (2015a). The human CD5L/AIM-CD36 axis: A novel autophagy inducer in macrophages that modulates inflammatory responses. Autophagy 11, 487-502.
NPL10: Sanjurjo, L., Aran, G., Roher, N., Valledor, A. F., and Sarrias, M. R. (2015b). AIM/CD5L: a key protein in the control of immune homeostasis and inflammatory disease. J Leukoc Biol 98, 173-184.
NPL11: Sarrias, M. R., Farnós, M., Mota, R., Sánchez-Barbero, F., Ibáñez, A., Gimferrer, I., Vera, J., Fenutría, R., Casals, C., Yélamos, J., et al. (2007). CD6 binds to pathogen-associated molecular patterns and protects from LPS-induced septic shock. Proc Natl Acad Sci USA 104, 11724-11729.
NPL12: Sarrias, M. R., Roselló, S., Sánchez-Barbero, F., Sierra, J. M., Vila, J., Yélamos, J., Vives, J., Casals, C., and Lozano, F. (2005). A role for human Sp alpha as a pattern recognition receptor. J Biol Chem 280, 35391-35398.

1-21. (canceled)

22. A recombinant human CD5L protein characterized in that it comprises the sequence between amino acids Ser20 and Gly347 of human CD5L fused to an 8-His tag sequence, as set forth in SEQ ID No. 3.

23. An active fragment derived from the recombinant human CD5L protein disclosed in claim 22 characterized in that it comprises amino acids Ser20 to Pro127 of human CD5L or amino acids Ser132 to Pro241 of human CD5L or amino acids Asp240 to Gly347 of human CD5L, fused to an 8-His tag sequence, as set forth in SEQ ID Nos. 4 to 6.

24. A preparation method of the recombinant human CD5L protein or the active fragment derived from the recombinant human CD5L protein of claim 22, characterized in that it uses the protein-expression system of a microbial or mammalian source, wherein the recombinant protein or an active fragment derived thereof is incorporated into a gene construct or into expression vectors directed to the transfection and expression of the specific polynucleotides.

25. A peptide derived from the recombinant human CD5L protein disclosed in claim 22 characterized in that it comprises 11 mer peptides within SRCR domains 1, 2 or 3 of the human CD5L, as set forth in SEQ ID Nos. 7 to 9.

26. A preparation method of the peptides derived from the recombinant human CD5L protein disclosed in claim 22 characterized in that the peptides are synthetically produced under GMP conditions.

27. A nucleic acid characterized in that it comprises a nucleotide sequence encoding the recombinant human CD5L protein of claim 22, as set forth in SEQ ID No. 10.

28. A pharmaceutical composition characterized in that it comprises:
a therapeutically effective amount of an active principle selected from the group consisting of a recombinant human CD5L protein, according to claim 22, or one or more active fragments derived thereof, or one or more peptides derived thereof, or one or more nucleic acids encoding full-length mature human CD5L or active fragments thereof, and a suitable excipient, diluent or carrier, wherein the suitable excipient, diluent or carrier is selected from the group consisting of a stabilizing agent or combinations thereof, a surfactant or combinations thereof, a buffering agent or combinations thereof, and an antioxidant or combinations thereof.

29. The pharmaceutical composition according to claim 28 characterized in that it comprises from 0.1 to 5.0% (by weight) of the active principle.

30. The pharmaceutical composition according to claim 28, characterized in that it comprises from 2 to 10% (by weight) of a stabilizing agent or combinations of stabilizing agents, wherein the stabilizing agent is selected from one or more of the group consisting of di-saccharides such as sucrose or trehalose, sugar alcohols such as mannitol, amino acids such as L-arginine or L-glycine or combinations thereof.

31. The pharmaceutical composition according to claim 28, characterized in that it comprises from 0.05 to 0.1% (by weight) of a surfactant or combinations of surfactants, wherein the surfactant is selected from one or more of the group consisting polysorbates, such as polysorbate 20 or polysorbate 80, polymers such as polyethylene glycol or combinations thereof.

32. The pharmaceutical composition according to claim 28, characterized in that it comprises from 0.2% to 0.5% (by weight) of a buffering agent or combinations of buffering agents, wherein the buffering agent is selected from one or more of the group consisting of citrates, phosphates or succinates.

33. The pharmaceutical composition according to claim 28, characterized in that it comprises from 0.01 to 0.04% (by weight) of an antioxidant or combinations of antioxidants, wherein the antioxidant is selected from one or more of the group consisting of L-glutathione, L-cysteine or L-methionine or combinations thereof.

34. The pharmaceutical composition according to claim 28, characterized in that it is compatible with one or more routes of administration of the group consisting of epicutaneous, subcutaneous, intramuscular and intravenous administrations.

35. The pharmaceutical composition according to claim 28 characterized in that it is in a liquid form, or in a solid form for dilution in water.

36. The pharmaceutical composition according to claim 28, characterized in that it is for use in the treatment of pathological conditions that comprise an intense inflammatory response, acute infections, sepsis or a cytokine storm, wherein the pathological conditions are selected from one or more of the group consisting of cardiovascular diseases, infectious diseases, parasite diseases, atherosclerosis, type 2 diabetes, rheumatoid arthritis, cancer or immunotherapy, hepatitis due to viral infection or alcohol, acute lung respiratory distress syndrome, cystic fibrosis, chronic obstructive pulmonary disease (COPD), asthma, acute dermatitis, severe acute respiratory syndrome (SARS) and Coronavirus diseases.


SEQUENCE LISTING (10 sequences)

SEQ ID NO: 1 - protein, 32 aa, Artificial Sequence
  Forward primer
CGTCGCTAGCGCATCTCCATCTGGAGTGCGGC

SEQ ID NO: 2 - protein, 30 aa, Artificial Sequence
  Reverse primer
CAAGGATCCTCCTGAGCAGATGACAGCCAC

SEQ ID NO: 3 - protein, 339 aa, Homo sapiens
SPSGVRLVGGLHRCEGRVEVEQKGQWGTVCDDGWDIKDVAVLCRELGCGAASGTPSGILYEPPAEKEQKVLIQSVSCTGTEDTLAQCEQEEVYDCSHDEDAGASCENPESSFSPVPEGVRLADGPGHCKGRVEVKHQNQWYTVCQTGWSLRAAKVVCRQLGCGRAVLTQKRCNKHAYGRKPIWLSQMSCSGREATLQDCPSGPWGKNTCNHDEDTWVECEDPFDLRLVGGDNLCSGRLEVLHKGVWGSVCDDNWGEKEDQVVCKQLGCGKSLSPSFRDRKCYGPGVGRIWLDNVRCSGEEQSLEQCQHRFWGFHDCTHQEDVAVICSGGSSHHHHHHHH

SEQ ID NO: 4 - protein, 119 aa, Homo sapiens
SPSGVRLVGGLHRCEGRVEVEQKGQWGTVCDDGWDIKDVAVLCRELGCGAASGTPSGILYEPPAEKEQKVLIQSVSCTGTEDTLAQCEQEEVYDCSHDEDAGASCENPGSSHHHHHHHH

SEQ ID NO: 5 - protein, 121 aa, Homo sapiens
SPVPEGVRLADGPGHCKGRVEVKHQNQWYTVCQTGWSLRAAKVVCRQLGCGRAVLTQKRCNKHAYGRKPIWLSQMSCSGREATLQDCPSGPWGKNTCNHDEDTWVECEDPGSSHHHHHHHH

SEQ ID NO: 6 - protein, 119 aa, Homo sapiens
DPFDLRLVGGDNLCSGRLEVLHKGVWGSVCDDNWGEKEDQVVCKQLGCGKSLSPSFRDRKCYGPGVGRIWLDNVRCSGEEQSLEQCQHRFWGFHDCTHQEDVAVICSGGSSHHHHHHHH

SEQ ID NO: 7 - protein, 11 aa, Homo sapiens
GRVEVEQKGQW

SEQ ID NO: 8 - protein, 11 aa, Homo sapiens
GRVEVKHQNQW

SEQ ID NO: 9 - protein, 11 aa, Homo sapiens
GRLEVLHKGVW

SEQ ID NO: 10 - DNA, 1166 bp, Homo sapiens
gatgctcgatatacgactcactatagggagacccaagctggctagcgtttaaacttaagcttatggctctgctattctccttgatccttgccatttgcaccagacctggattcctagcgtctccatctggagtgcggctggtggggggcctccaccgctgtgaagggcgggtggaggtggaacagaaaggccagtggggcaccgtgtgtggctacggctgggacattaaggacgtggctgtgttgtgccgggagctgggctgtggagctgccagcggaacccctagtggtattttgtatgagccaccagcagaaaaagagcaaaaggtcctcatccaatcagtcagttgcacaggaacagaagatacattggctcagtgtgagcaagaagaagtttatgattgttcacatgatgaagatgctggggcatcgtgtgagaacccagagagctctttctccccagtcccagagggtgtcaggctggctgacggccctgggcattgcaagggacgcgtggaagtgaagcaccagaaccagtggtataccgtgtgccagacaggctggagcctccgggccgcaaaggtggtgtgccggcagctgggatgtgggagggctgtactgactcaaaaacgctgcaacaagcatgcctatggccgaaaacccatctggctgagccagatgtcatgctcaggacgagaagcaacccttcaggattgcccttctgggccttgggggaagaacacctgcaaccatgatgaagacacgtgggtcgaatgtgaagatccctttgacttgagactagtaggaggagacaacctctgctctgggcgactggaggtgctgcacaagggcgtatggggctctgtctgtgatgacaactggggagaaaaggaggaccaggtggtatgcaagcaactgggctgtgggaagtccctctctccctccttcagagaccggaaatgctatggccctggggttggccgcatctggctggataatgttcgttgctcaggggaggagcagtccctggagcagtgccagcacagattttgggggtttcacgactgcacccaccaggaagatgtggctgtcatctgctcaggaggatcctcccatcaccatcaccatcaccatcactgataatctagaggcccgtagccggatgcg